(19) United States
(12) Patent Application Publication
(10) Pub. No.: US 2026/0041401 A1
(43) Pub. Date: Feb. 12, 2026

(54) ULTRASOUND IMAGING SYSTEM AND METHOD FOR VOLUMETRIC PARAMETRIC IMAGING IN CONTRAST ENHANCED ULTRASOUND IMAGING

(71) Applicant: KONINKLIJKE PHILIPS N.V., EINDHOVEN (NL)

(72) Inventors: Shiying Wang, Melrose, MA (US); Thanasis Loupas, Kirkland, WA (US); William Tao Shi, Wakefield, MA (US); Mingxin Zheng, Cambridge, MA (US)

(21) Appl. No.: 19/099,134

(22) PCT Filed: Jul. 25, 2023
(86) PCT No.: PCT/EP2023/070495

§ 371 Date: Jan. 28, 2025

Related U.S. Application Data
(60) Provisional application No. 63/393,324, filed on Jul. 29, 2022.

(51) Int. Cl. A61B 8/00

(52) U.S. Cl. CPC A61B 8/481; A61B 8/467

(57) ABSTRACT

A system, method (200) and a tangible, non-transitory computer readable medium (130) that stores instructions are described. When executed by a processor (120) the instructions cause the processor to: determine a color value of a contrast agent as a function of a selected time-based parameter for each pixel (202); determine an opacity value of the contrast agent for each pixel (204); and a display (140) in communication with the processor and configured to display (206) the color value, and the opacity value for each pixel.

### BACKGROUND

Contrast-enhanced ultrasound (CEUS) is an ultrasound imaging technique used in a variety of clinical applications. CEUS can detect the nonlinear signals received from microbubbles which circulate in the blood stream after an intravenous injection of an ultrasound contrast agent. As such CEUS imaging allows for documentation of tissue perfusion due to comparatively slow flow at the capillary level, as well as visualizing blood flow in arteries and veins. As a result, CEUS is capable at providing dynamic visualization of blood flow at both the macro-and micro-circulation levels. Among other clinical applications, CEUS imaging mode is recommended in the diagnosis and treatment monitoring of lesions on the liver, which may be malignant.

Parametric imaging with CEUS has been frequently used to characterize blood perfusion patterns and consequently the lesion type. For example, many lesions such as benign focal nodular hyperplasia (FNH) and malignant hepatocellular carcinoma (HCC) exhibit rapid complete enhancement during the arterial phase. However, FNHs exhibit an “early arrival” spoke-wheel arterial enhancement pattern, whereas HCCs exhibit a “spatially complex” chaotic arterial enhancement pattern. The time-of-arrival (TOA) parametric imaging, which can directly visualize the time taken for the contrast agent to arrive in the target tissue, is particularly effective to delineate arterial enhancement patterns such as the time-sensitive spoke-wheel.

However, currently available parametric imaging in CEUS is limited to two dimensional (2D) imaging. Three-dimensional (3D) and four-dimensional (4D=3D data+time) CEUS parametric imaging modes are desired and advantageous to visualize various aspects of the anatomy, such as the vascular tortuosity of malignant lesions (e.g., HCC) as well as the center-to-rim blood supply pattern in benign tumors (e.g., FNH).

What is needed is a system and method that overcomes at least the noted drawbacks of known systems set forth above.

### SUMMARY

According to one aspect of the present disclosure, a system for providing ultrasound images is described. The system comprises: a source of ultrasound signal data from a contrast-enhanced ultrasound (CEUS) examination; a processor; a tangible, non-transitory computer-readable medium that stores instructions, which when executed by the processor cause the processor to: determine a color value of a contrast agent as a function of a selected time-based parameter for each pixel; determine an opacity value of the contrast agent for each pixel; and a display in communication with the processor and configured to display the color value and the opacity value for each pixel.

According to another aspect of the present disclosure, a tangible, non-transitory computer-readable medium that stores instructions is disclosed. When executed by a processor, the instructions cause the processor to: determine a color value of a contrast agent as a function of a selected time-based parameter for each pixel; determine an opacity value of the contrast agent for each pixel; and display the color and opacity value for each pixel.

According to yet another aspect of the present disclosure, a method of providing ultrasound images is disclosed. The method comprises: determining a color value of a contrast agent as a function of a selected time-based parameter for each pixel; determining an opacity value of the contrast agent for each pixel; and displaying the color value, and the opacity value for each pixel.

### BRIEF DESCRIPTION OF THE DRAWINGS

The representative embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a CEUS imaging system for imaging a portion of a body, according to a representative embodiment.

FIG. 2A is a flow chart of a method of assigning a color value, an intensity value and an opacity value (α) of the contrast agent as a function of the selected time-based parameter according to a representative embodiment.

FIG. 2B is a flow chart of the method of FIG. 2A depicting various tools for assigning a color value, an intensity value and an opacity value (α) of the contrast agent as a function of the selected time-based parameter in accordance with a representative embodiment.

FIG. 3A is a graph of color versus time of arrival (TOA) of a contrast agent according to a representative embodiment.

FIG. 3B is a graph of opacity versus contrast agent intensity according to a representative embodiment.

FIG. 3C is a depiction of a display of a CEUS imaging system of a representative embodiment depicting the color and opacity of FIGS. 3A-3B for each pixel of the display.

FIG. 4A is a graph of color versus time of arrival (TOA) of a contrast agent according to a representative embodiment.

FIG. 4B is a graph of opacity versus TOA according to a representative embodiment.

FIG. 4C is a depiction of a display of a CEUS imaging system of a representative embodiment depicting the color and opacity of FIGS. 4A-4B for each pixel of the display.

FIG. 5A is a graph of color versus time of arrival (TOA) of a contrast agent according to a representative embodiment.

FIG. 5B is a graph of opacity versus contrast agent intensity and TOA according to a representative embodiment.

FIG. 5C is a depiction of a display of a CEUS imaging system of a representative embodiment depicting the color and opacity of FIGS. 5A-5B for each pixel of the display.

FIGS. 6A-6I are depictions of a display of a CEUS imaging system of a representative embodiment depicting the color and opacity of FIGS. 5A-5B for each pixel of the display according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

As used in the specification and appended claims, and in addition to their ordinary meanings, the term ‘approximately’ mean to with acceptable limits or degree. For example, “approximately 20 GHz” means one of ordinary skill in the art would consider the signal to be 20 GHz within reasonable measure.

As used in the specification and appended claims, in addition to their ordinary meanings, the term ‘substantially’ means within acceptable limits or degree. For example, the “plurality of transducer ports are substantially the same” means one of ordinary skill in the art would consider the plurality of transducer ports to be the same.

As used herein, the term “opacity” refers to the degree of blocking the background pixel color and displaying the current pixel color. More generally, the present teachings contemplate use of a known color model commonly referred to as an RGBA model where RGBA stands for red green blue alpha, where alpha is the opacity value of the pixel. While it is sometimes described as a color space, it is actually a three-channel RGB color model supplemented with a fourth alpha (α) channel for opacity. As described in connection with various representative embodiments, the value of alpha indicates how opaque each pixel is and allows an image to be combined over others using alpha compositing with transparent areas and anti-aliasing of the edges of opaque regions. The opacity (α) has a value between 0 and 1. Alternatively, the transparency value could be assigned to each pixel, where the transparency refers to the degree of displaying the current pixel color at a particular pixel, and blocking the background color of the pixel. The transparency (T) has a value between 0 and 1, with T=1−α. When α=0, T=1, the pixel is fully transparent, and all background colors will be displayed with no blocking from the current pixel; however, no current pixel color will be displayed since it is transparent. Simply speaking, the human eye can see the background pixel without seeing the current pixel. By contrast, when α=1, T=0, the pixel is fully opaque. In this case, no background color will be displayed due to the complete blocking of the current pixel. The current pixel color will be displayed completely. Simply speaking, when α=1 the human eye cannot see the background pixel due to the blocking of the current pixel. Of course, assigning values of opacity (α) and transparency (T) between 0 and 1 results in varying degrees of opacity/transparency. It is emphasized that the RGB colors are merely illustrative, and other color schemes are contemplated. Just by way of example, the present teachings contemplate the use of a CMY color model where CMY stands for Cyan Magenta Yellow. Similarly, a CMYA model having the three colors that is supplemented by a fourth (α) channel is contemplated for use in connection with the various representative embodiments described herein.

As alluded to above, known parametric imaging is limited to 2D in CEUS. Specifically, in the 2D parametric imaging mode, parameters are derived from 2D CEUS frames and then color-coded to form the output images. There have been multiple methods for designs of color coding and mapping, for example, dynamic or adaptive color maps. In 3D/4D parametric imaging of the present teachings, time-based parameters are derived from 3D/4D CEUS volumes and then color-coded to form the output volumes on a display or monitor for review from a variety of orientations. While various 2D color mapping approaches can be applied directly to 3D/4D parametric imaging, among other beneficial improvements, one feature of 3D/4D parametric imaging of the present teachings is that transparencies of the output volume (3D or 4D) pixels can be controlled. As described more fully below, to visualize the color-coded anatomical features (e.g., arteries and/or veins) inside a 3D object (e.g., an organ, or a lesion or tumor), transparency values are beneficially adequately adjusted between 1 (fully transparent) and 0 (fully opaque). For example, to highlight the origin of perfusion, the transparency can be set to increase with TOA.

As described more fully below, the present teachings relate to a CEUS system, method and tangible, non-transitory computer readable to provide 4D parametric imaging in CEUS with a time-based parameter used as the color-coded imaging parameter. By the present teachings, a parameter is extracted from 3D/4D data of a CEUS scan. Parametric imaging provides the display of each image sequence as a single composite image in which each pixel is color-coded and has an opacity/transparency value to characterize a time-based parameter of the contrast media dynamics to provide direct visual evaluation of vascular features of an anatomical part (to include lesions or tumors) over a particular CEUS image sequence/loop. In accordance with a representative embodiment, the time-based parameter is illustratively the time of arrival (TOA) of the contrast agent at a location of anatomical interest. The TOA is merely illustrative, and is employed as an exemplary feature because of both its comparatively straightforward formulation from a time-intensity curve, and its weak dependency on contrast medium infusion conditions. However, other time-based parameters are contemplated for implementation in accordance with various representative embodiments of the present teachings. These parameters are often of interest in the analysis of anatomical features using CEUS imaging. These parameters include, but are not limited to: a time to peak of the contrast agent; a wash-in rate of the contrast agent; a rise time of the contrast agent; a peak intensity of the contrast agent; a wash-out rate of the contrast agent; a mean transit time of the contrast agent; a fall time of the contrast agent; and an area under a curve of the contrast agent for each pixel; and a slope or change in the slope of an intensity of the contrast agent at each pixel. Accordingly, the present teachings contemplate a variety of time-dependent parameters for implementation in 4D CEUS parametric imaging and diagnosis applications.

Among other benefits, the workflow according to the present teachings allows improved viewing of the results of an ultrasound scan. Notably, not only are the various pixels of a display of a CEUS system color coded to reflect a particular time-based parameter (e.g., TOA), but also the opacity (and thus transparency) of each pixel in a 3D or 4D image is provided. The images are color-coded (e.g., RGB, CMY) and depict the opacity (α) of the selected time-based parameter in each pixel of the display. As such, each pixel of the display is assigned a color and an opacity value, and by assigning the opacity value (or transparency value) at each pixel, improvement in the visualization of the selected time-based parameter is realized. The resultant image comprised of a color code and transparency value at each pixel is configured to be rotated 360° around an axis to allow review of this time-based parameter from a different vantage point or viewing angle. This ability to rotate the image allows review of different degrees of opacity/transparency and improves the accuracy of the review by a trained clinician in a diagnostic application. Accordingly, the present teachings enable the 3D or 4D visualization of the selected time-based parameter on a display.

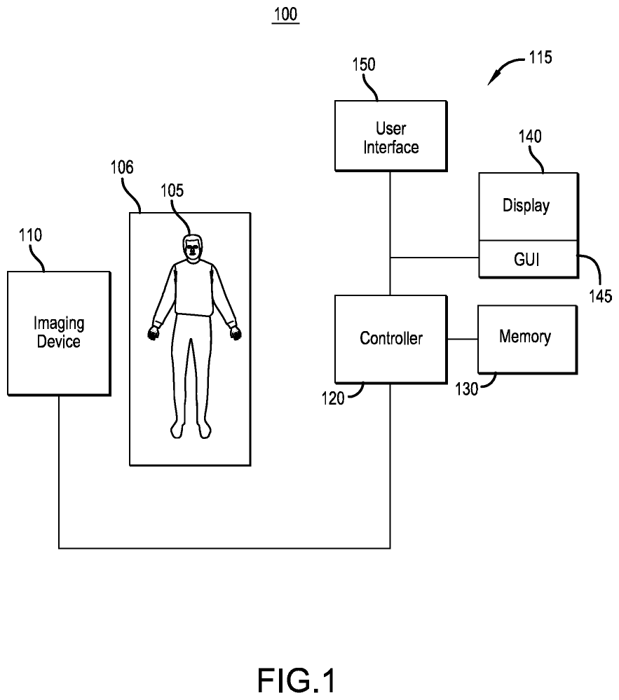

FIG. 1 is a simplified block diagram of an ultrasound imaging system 100 for imaging a region of interest of a subject, according to a representative embodiment.

Referring to FIG. 1, the ultrasound imaging system 100 comprises an imaging device 110 and a computer system 115 for controlling imaging of a region of interest in a patient, for example in a patient 105 on a table 106. The imaging device 110 is illustratively an ultrasound imaging system capable of providing a contrast-enhanced ultrasound (CEUS) image scan of a region of interest (ROI) of the patient 105.

The computer system 115 receives image data from the imaging device 110, and stores and processes the imaging data according to representative embodiments described herein. The computer system 115 comprises a controller 120, a memory 130, a display 140 which may in turn comprise a graphical user interface (GUI) 145, and a user interface 150. The display 140 may also include a loudspeaker (not shown) to provide audible feedback. Notably, and as described more fully below, one significant paramter in CEUS imaging is the time of arrival (TOA) of the contrast agent at a particular location of the anatomy of the patient. Among many control functions afforded by the GUI 145 and the user interface 150, in accordance with a representative embodiment the TOA is measured from a start time of the injection of the contrast agent, which may be controlled from the GUI 145 or the user interface 150.

The memory 130 stores instructions executable by the controller 120. When executed, and as described more fully below, the instructions cause the controller 120 to allow the user to perform different steps using the GUI 145 or the user interface 150, or both, and to initialize an ultrasound imaging device comprising a transducer. Notably, and among other functions, the GUI 145 and the display 140, or the user interface 150 and the display 140, are used to select the desired time-based parameter desired to be reviewed by the clinician or sonographer. In addition, the controller 120 may implement additional operations based on executing instructions, such as instructing or otherwise communicating with another element of the computer system 115, including the memory 130 and the display 140, to perform one or more of the above-noted processes.

The memory 130 may include a main memory and/or a static memory, where such memories may communicate with each other and the controller 120 via one or more buses. The memory 130 stores instructions used to implement some or all aspects of methods and processes described herein.

The memory 130 may be implemented by any number, type and combination of random access memory (RAM) and read-only memory (ROM), for example, and may store various types of information, such as software algorithms, which serves as instructions, which when executed by a processor cause the processor to perform various steps and methods according to the present teachings. Furthermore, updates to the methods and processes described herein may also be provided to the computer system 115 and stored in memory 130.

The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 130 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 may store software instructions and/or computer readable code that enable performance of various functions. The memory 130 may be secure and/or encrypted, or unsecure and/or unencrypted.

“Memory” is an example of computer-readable storage media, and should be interpreted as possibly being multiple memories or databases. The memory or database for instance may be multiple memories or databases local to the computer, and/or distributed amongst multiple computer systems or computing devices, or disposed in the ‘cloud’ according to known components and methods. A computer readable storage medium is defined to be any medium that constitutes patentable subject matter under 35 U.S. C. § 101 and excludes any medium that does not constitute patentable subject matter under 35 U.S. C. § 101. Examples of such media include non-transitory media such as computer memory devices that store information in a format that is readable by a computer or data processing system. More specific examples of non-transitory media include computer disks and non-volatile memories.

The controller 120 is representative of one or more processing devices, and is configured to execute software instructions stored in memory 130 to perform functions as described in the various embodiments herein. The controller 120 may be implemented by field programmable gate arrays (FPGAs), application specific integrated circuits (ASICs), systems on a chip (SOC), a general purpose computer, a central processing unit, a computer processor, a microprocessor, a graphics processing unit (GPU), a microcontroller, a state machine, programmable logic device, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Modules have software instructions to carry out the various functions using one or multiple processors that may be within the same computing device or which may be distributed across multiple computing devices.

The display 140 may be a monitor such as a computer monitor, a television, a liquid crystal display (LCD), a light emitting diode (LED) display, a flat panel display, a solid-state display, or a cathode ray tube (CRT) display, or an electronic whiteboard, for example. The display 140 may also provide a graphical user interface (GUI) 145 for displaying and receiving information to and from the user.

The user interface 150 may include a user and/or network interface for providing information and data output by the controller 120 and/or the memory 130 to the user and/or for receiving information and data input by the user. That is, the user interface 150 enables the user to operate the imaging device as described herein, and to schedule, control or manipulate aspects of the ultrasound imaging system 100 of the present teachings. Notably, the user interface 150 enables the controller 120 to indicate the effects of the user's control or manipulation. The user interface 150 may include one or more of ports, disk drives, wireless antennas, or other types of receiver circuitry. The user interface 150 may further connect one or more interface devices, such as a mouse, a keyboard, a mouse, a trackball, a joystick, a microphone, a video camera, a touchpad, a touchscreen, voice or gesture recognition captured by a microphone or video camera, for example.

Notably, the controller 120, the memory 130, the display 140, the GUI 145 and the user interface 150 may be located away from (e.g., in another location of a building, or another building) the imaging device 110 operated by a sonographer. The controller 120, the memory 130, the display 140, the GUI 145 and the user interface 150 may be, for example, located where the radiologist/clinician is located. Notably, however, additional controllers, the memories, displays, GUI and user interfaces may be located near the sonographer and are useful in effecting the various functions of the imaging device 110 needed to complete the CEUS scans contemplated by the present teachings. Finally, in a more general sense, the ultrasound imaging system 100 may comprise a source of ultrasound signal data from a CEUS examination. In such an embodiment, the computer system 115 may be connected to the source of these ultrasound signal data (e.g., at a remote location from the computer system 115) from the CEUS examination to receive these data, but the ultrasound imaging system 100 does not include the imaging device 110 or the table 106.

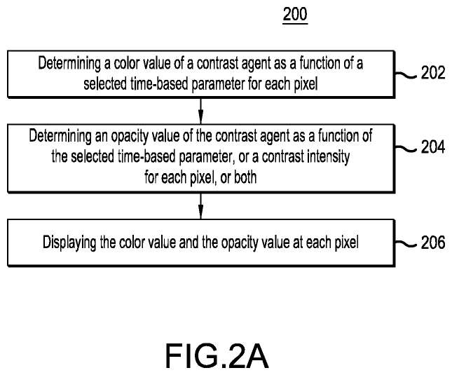

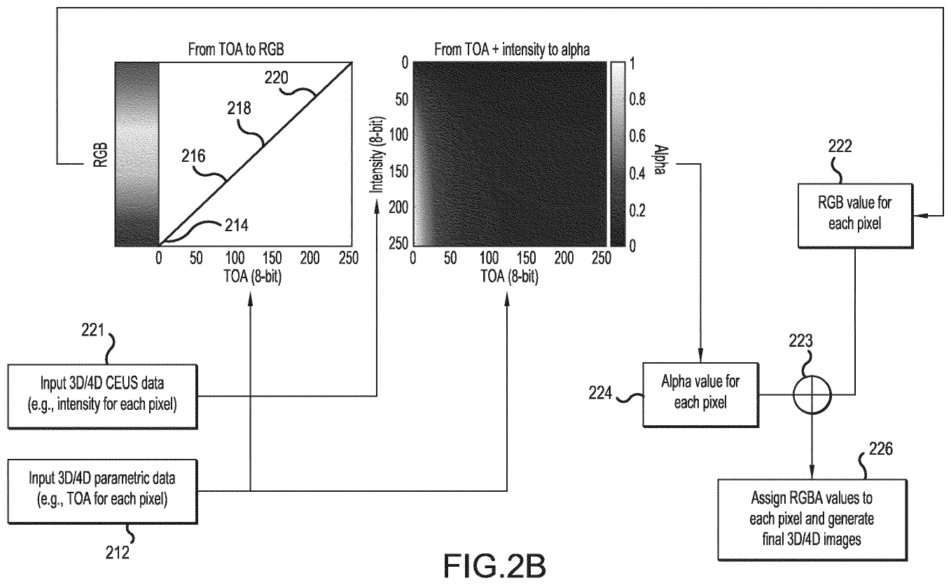

FIG. 2A is a flow chart of a method 200 of collecting images using the CEUS imaging system of FIG. 1 to provide images that are color-coded based on the time-based parameter and the opacity a (and thus, as noted above, the transparency, T, where α=1−T) for each pixel on the display 140 according to a representative embodiment. Various aspects and details of the method are common to those described in connection with representative embodiments of FIG. 1. These common aspects and details may not be repeated to avoid obscuring the presently described representative embodiment.

At 202, the method begins with determininga color value of a contrast agent-enhanced ultrasound signal to each pixel of the display 140 as a function of the selected time-based parameter. After color values for each pixel are determined, each color value is assigned to its particular pixel of the display 140 by the controller 120. As noted above, TOA provides an excellent time-based parameter for describing the various aspects of the method 200. So, following this example, and as described more fully below, a color value (RGB or CMY) is first determined and then and assigned to each pixel based on the TOA of the contrast agent in the body associated with that pixel. However, and at the risk of redundancy, it is noted the color value assigned to each pixel may be based on one of a number of other time-based parameters useful in diagnosing a condition based on the US images acquired by the ultrasound imaging system 100. Notably, the determination of the color of each pixel can be a more intuitive and efficient way to quantify the TOA values. Instead of reading the TOA values of each pixel, a Red color can indicate early arrived contrast and a Blue color can indicate late arrived contrast.

At 204, the method continues with determining an opacity value of the contrast agent to each pixel of display 140. After the opacity values are determined, each opacity value is assigned to its particular pixel of the display 140 by the controller 120.

As described more fully below, the assignment of the contrast agent may be carried out as a function of the selected time-based parameter, or an intensity of the contrast agent (contrast intensity) for each pixel of the display, or both. The assigning of the opacity (and thereby the transparency) beneficially allows review of an anatomical feature more clearly, with less opacity (greater transparency) in certain locations of study. Notably, and as described more fully below, the ability to rotate the image on the display 140 using the GUI 145 or the user interface 150 allows the review of the 3D/4D image from different vantage points. As such, by assigning an opacity value to each pixel based on the selected time-based parameter, or contrast intensity for each pixel of the display, or both, allows the clinician to see through the 3D/4D image to a point of interest by assigning a low opacity (high transparency) value to certain pixels in a region of interest between the surface of the anatomical part to enable other pixels beneath this surface in the region of interest. Alternatively, the method may assign a higher opacity (low transparency) value to another portion of the anatomical part in a region of interest so that features on a surface in the region of interest may be studied.

As noted above, and as described more fully below, the opacity value assigned to each pixel of the display 140 may be effected as a function of the time-dependent parameter selected for the particular CEUS exam. So, continuing the example, the opacity value may be assigned based on the TOA. Just by way of illustration, each pixel in a region of interest of the anatomical part being examined may be assigned an opacity value based on the TOA to facilitate review of the region of interest from a wide range (e.g., 360°) of viewing angles/vantage points of the 3D/4D image provided to the display. Notably, the determination of the opacity value to be assigned to each pixel is preferably done by optimizing the opacity value assigned to each pixel to achieve the best imaging quality for better diagnostic accuracy and confidence. Specifically, the opacity cannot be too low to lose the color value for the current pixel; and it cannot be too high to block the colors from background pixels. Thus some embodiments may restrict the range of opacities that are used for setting the pixel value to a range between full transparency and full opacity.

Alternatively, and as described more fully below, the opacity value assigned to each pixel of the display 140 may be effected as a function of the contrast intensity at each pixel. The opacity value can be calculated as a function of the CEUS intensity at a given pixel (for example, a value from 0 to 1 derived from a nonlinear mapping of the CEUS intensity like an S-curve, for example, as well as a function of the CEUS intensities from the start of the acquisition up to the current volume number (e.g., the temporal mean, minimum, maximum, the temporal standard deviation, to name a few).

Still alternatively, the opacity value assigned to each pixel of the display 140 may be effected as a function of both the time-dependent parameter selected for the particular CEUS exam, and the contrast intensity at each pixel. So, continuing the example, the opacity value may be assigned based on the TOA and the contrast intensity at each pixel of the display 140. Just by way of illustration, each pixel in a region of interest of the anatomical part being examined may be assigned an opacity value based on the TOA and the contrast intensity to facilitate review of the region of interest from a wide range (e.g., 360°) of viewing angles/vantage points of the 3D/4D image provided to the display.

At 206, the method 200 continues with the displaying of the color value and the opacity value assigned in 202, 204 on the display 140 for review by the clinician or sonographer. Again, as described more fully below, the assignment of both a color value at each pixel based on the selected time-dependent parameter, and the opacity value at each pixel as a function of the time-based parameter and/or the contrast intensity facilitates review of various aspects of the anatomical part being examined to allow review from different angles to reveal elements beneath a surface of the anatomical part, and/or on the surface of the anatomical part, and/or above the surface of the anatomical part.

FIG. 2B is a flow chart of the method 200 of collecting images using the CEUS imaging system of FIG. 1 to provide images that are color-coded based on the time-based parameter and the opacity a (and thus the transparency, T, where α=1−T) for each pixel on the display 140 according to a representative embodiment. The flow chart of FIG. 2B highlights and depicts various aspects of the method 200 described in connection with FIG. 2A. Various aspects and details of the method are common to those described in connection with representative embodiments of FIGS. 1-2A. These common aspects and details may not be repeated to avoid obscuring the presently described representative embodiment.

At 212 the method depicts assigning a color value of a contrast agent to each pixel of the display 140 as a function of the selected time-based parameter as described in connection with 202 in FIG. 2A. Again, in the presently described representative embodiment, the time-dependent parameter is TOA. As shown, the assignment of a color to a particular data point varies linearly with the TOA, with the first arriving contrast data being coded red (R), which varies in color illustratively across the visible spectrum with time from red to blue. So, at point 214 the color assigned to the contrast agent that arrives in the first few seconds is red, and the pixels on the display 140 that depict the contrast agent in these first few seconds as red.

Similarly, contrast agent that arrives at approximately 100 seconds at 216 is assigned a color yellow, and the pixels on the display that depict the contrast agent arriving at 100 seconds are yellow. Contrast agent that arrives at approximately 150 seconds at 218 is assigned a color green, and the pixels on the display that depict the contrast agent arriving at 100 seconds are green.

Contrast agent that arrives at approximately 150 seconds at 220 is assigned a color blue, and the pixels on the display that depict the contrast agent arriving at 100 seconds are blue. Notably, the variation of shade of the colors of the visible spectrum can be selected to identify the TOA of contrast agent with significant granularity and thus precision. Moreover, based on the color assigned to a particular TOA, clinical information related to the perfusion pattern of the contrast agent is shown offering a better understanding of the timing of the perfusion pattern with different levels or depths of the anatomical structure due to assignment of color and opacity values to each pixel of the display 140. Finally, it is again emphasized that TOA is merely an illustrative time-based parameter, and other time-based parameters such as described above, are contemplated for use to provide parametric imaging that results in the display of each image sequence as a single composite image in which each pixel is color-coded to characterize a time-based parameter of the contrast media dynamics to provide direct visual evaluation of vascular features of an anatomical part (to include lesions or tumors) over a particular CEUS image sequence/loop.

At 212 one aspect of 204 is depicted with the assignment of one component of the opacity value (α) of each pixel. Specifically, the parametric imaging provides the display of each image sequence as a single composite image in which each pixel has an opacity/transparency value to characterize a time-based parameter (in this example TOA) of the contrast media dynamics. As such 212 depicts the assignment of a TOA value to an opacity/transparency value. Accordingly, in the presently described representative embodiment, the assigned color changes from comparatively opaque (i.e., showing its originally assigned color) for the earliest arriving contrast agent and depicts the lowest transparency (T=1−α=0)/highest opacity (α=1), whereas later arriving contrast agent is assigned, for example a red, orange or yellow shade of color with decreasing opacity/increasing transparency as shown.

At 221 another aspect of 204 is depicted with the assignment of another component of the opacity value (α) of each pixel. Specifically, the parametric imaging provides the display of each image sequence as a single composite image in which each pixel has an opacity/transparency value to characterize a contrast intensity of the contrast media dynamics. As such, 212 depicts the assignment of a contrast intensity value to an opacity/transparency value. Accordingly, in the presently described representative embodiment, the assigned color changes from comparatively opaque for the highest intensity contrast agent and depicts the lowest transparency (T=1−α=0)/highest opacity (α=1), whereas a lower intensity contrast agent is assigned, for example a red, orange or yellow shade of color with decreasing opacity/increasing transparency as shown.

Accordingly, for each pixel of the displayed image, 212 and 221 provide a color coded value based on TOA and an opacity value as a function of TOA or contrast intensity, or both.

At 223 the color (RGB) value for each pixel is added to the opacity value at 224 to assign at 226, for each pixel in the displayed CEUS image, a color coded RGB and opacity (RGBA) value. Accordingly, and as described more fully below, assignment of both a color value at each pixel based on the selected time-dependent parameter, and the opacity value at each pixel as a function of the time-based parameter and/or the contrast intensity facilitates review of various aspects of the anatomical part being examined to allow review from different angles to reveal elements beneath a surface of the anatomical part, and/or on the surface of the anatomical part, and/or above the surface of the anatomical part.

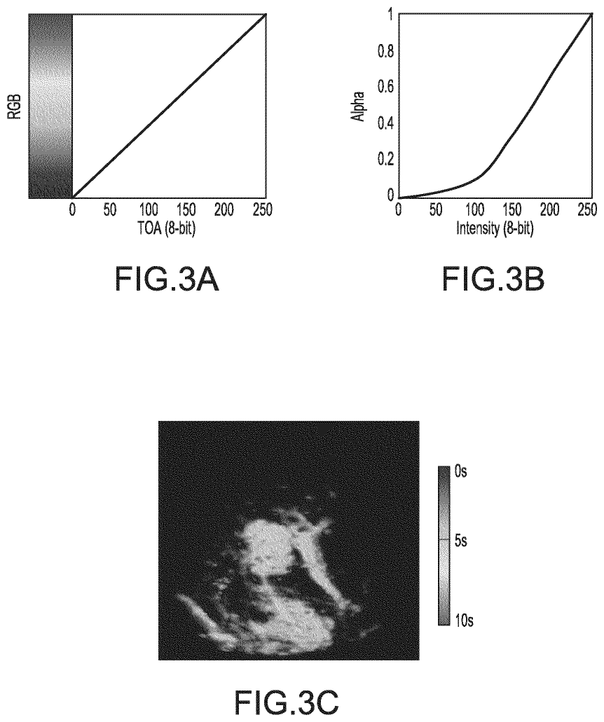

Turning to FIGS. 3A-3C assignment of color and opacity as a function of TOA to pixels on a display are described. Various aspects and details of the method are common to those described in connection with representative embodiments of FIGS. 1-2B. These common aspects and details may not be repeated to avoid obscuring the presently described representative embodiment.

FIG. 3A shows a graph of color versus time of arrival (TOA) of a contrast agent according to a representative embodiment. As described above, according to one representative embodiment of the method (or algorithm), the assignment of a color to a particular data point varies linearly with the TOA, with the first arriving contrast data being coded red (R), whereas a particular data point from the source of ultrasound image data that arrives at a latest time is color coded blue (B). Again, the other colors of the visible spectrum between red and blue are assigned to data points arriving at times between the first (R) data point and the last (B) data point being assigned other colors (orange, yellow, green) along the spectrum of colors. Moreover, as noted above, the shading of the colors of the rainbow along the spectrum can be used to further discern the TOAs of the data points from the current CEUS scan by gradation of these shades with more shades providing greater distinction between TOA of data points represented by the color of the pixel. As such, the gradation of the shades of the colors of the visible spectrum between red and blue can be selected to identify the TOA of contrast agent with significant granularity and thus precision.

FIG. 3B is a graph of opacity versus contrast agent intensity according to a representative embodiment. As shown, in the present method (or algorithm), the assignment of an opacity value is not linear, but rather somewhat parabolic and increasing with contrast intensity. As such, data points having a comparatively low magnitude of contrast intensity are accorded a low opacity/high transparency, whereas data points having a comparatively high magnitude are accorded a comparatively high opacity/low transparency. Just by way of illustration, a data point from a CEUS scan that has a contrast intensity of 50 on the relative scale of FIG. 3B has an opacity of approximately 0.05. More generally, the CEUS intensity is normalized and quantified by unsigned integers of 8 bits, having a range of 0 to 255.

Such a data point would be comparatively transparent. By contrast, a data point from the same CEUS scan that has a contrast intensity of 200 on the relative scale of the drawing has an opacity of approximately 0.75 or a transparency of approximately 0.25. Such a data point would be comparatively much less transparent. Generally, and as shown by example in FIG. 3B the relationship between the opacity and the intensity can be determined algorithmically, or by testing a large number of candidate shapes of the relationship to generate the results. The selected shapes are determined to provide sufficient image quality for improved diagnostic accuracy.

FIG. 3C is a depiction of a display of a CEUS imaging system of a representative embodiment depicting the color and opacity for each pixel of the display. Notably, the scale on the right of the drawing shows in gray scale the arrival time of data points from the CEUS with opacity values for each pixel being assigned based on the relationship of FIG. 3B. In this embodiment, by application of the relationships of FIGS. 3A and 3B to assign color and opacity of each data point, when depicted on a color display, the 3D TOA volume image has comparatively opaque colors on a surface of the anatomical part being viewed, making the discernment of internal structures of the anatomical part (e.g., lesion) more difficult. Moreover, although this effect of obscuring internal structures of the anatomy may be beneficial for some diagnoses, the effect may be less beneficial in others. So, while not an ideal method, the algorithm providing color and opacity values per FIGS. 3A-3B will enable review of the 3D/4D volume image of the anatomical part being studied in the CEUS imaging procedure.

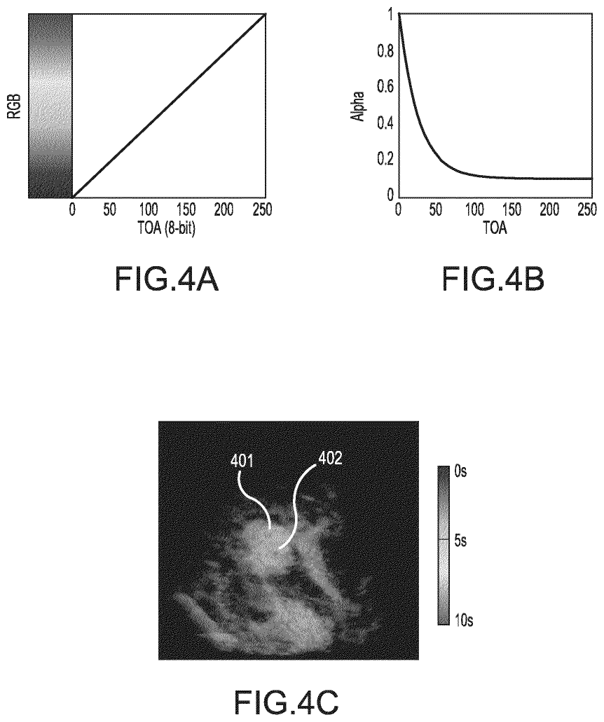

Turning to FIGS. 4A-4C assignment of color and opacity as a function of TOA to pixels on a display are described. Various aspects and details of the method are common to those described in connection with representative embodiments of FIGS. 1-3C. These common aspects and details may not be repeated to avoid obscuring the presently described representative embodiment.

FIG. 4A shows a graph of color versus time of arrival (TOA) of a contrast agent according to a representative embodiment. As described above, according to one representative embodiment of the method (or algorithm), the assignment of a color to a particular data point varies linearly with the TOA, with the first arriving contrast data being coded red (R), whereas a particular data point from the source of ultrasound image data that arrives at a latest time is color coded blue (B). Again, the other colors of the visible spectrum between red and blue are assigned to data points arriving at times between the first (R) data point and the last (B) data point being assigned other colors (orange, yellow, green) along the spectrum of colors. Moreover, as noted above, the shading of the colors of the rainbow along the spectrum can be used to further discern the TOAs of the data points from the current CEUS scan by gradation of these shades with more shades providing greater distinction between TOA of data points represented by the color of the pixel. As such, the gradation of the shades of the colors of the visible spectrum between red and blue can be selected to identify the TOA of contrast agent with significant granularity and thus precision.

FIG. 4B is a graph of opacity versus contrast agent intensity according to a representative embodiment. As shown, in the present method (or algorithm), the assignment of an opacity value again is not linear, but rather somewhat of a decreasing exponential function. As such, data points having a comparatively low TOA are accorded a high opacity/low transparency, whereas data points having a comparatively high TOA are accorded a comparatively low opacity/high transparency. The relative scale of the TOA is normalized and quantified by assigned integers of 8 bits having a range of 0 to 255, where 0 is assigned to the first arriving contrast, and 255 is assigned to the last arriving contrast. Just by way of illustration, a data point from a CEUS scan that has a contrast TOA of 10 on the relative scale of the drawing has an opacity of approximately 0.8. Such a data point would be comparatively opaque. By contrast, a data point from the same CEUS scan that has a contrast TOA of 200 on the relative scale of the drawing has an opacity of approximately 0.1 or a transparency of approximately 0.9. Such a data point would be comparatively much more transparent. Generally, and as shown by example in FIG. 4B the relationship between the opacity and the contrast TOA can be determined algorithmically, or by testing a large number of candidate shapes of the relationship to generate the results. The selected shapes are determined to provide sufficient image quality for improved diagnostic accuracy.

FIG. 4C is a depiction of a display of a CEUS imaging system of a representative embodiment depicting the color and opacity for each pixel of the display. Notably, the scale on the right of the drawing shows in gray scale the arrival time of data points from the CEUS with opacity values for each pixel being assigned based on the relationship of FIG. 4B. In this embodiment, by application of the relationships of FIGS. 4A and 4B to assign color and opacity of each data point, when depicted on a color display, the 3D TOA volume image has comparatively transparent colors on a surface of the anatomical part being viewed, making the discernment of internal structures of the anatomical part (e.g., lesion) better than in the image of FIG. 3C. However, and although not easily viewed in FIG. 4C, there are blotches 401, 402 of color (e.g., red) due to the comparatively high opacity at the beginning of the CEUS imaging procedure (i.e., from the start time of the injection of the contrast agent as noted above). Although the obscuring of internal structures of the anatomy in the image by blotches 401, 402 may be beneficial in some diagnoses, their impact may be less beneficial in others. Notably, residual clutter, early arriving contrast agent values, or background noise all have comparatively low TOA values, and can create obstacles on the outer surfaces of the anatomical part being imaged, rendering review of the internal structure of the anatomical portion more difficult.

Moreover, the desirably more clear structure of the outer surface of the anatomical part is often covered at least in part by the early arriving contrast agent to the image. However, even in view of these drawbacks, the method described in connection with FIGS. 4A-4C provides improvement in the resultant view of the 3D/4D volume image of FIG. 4C.

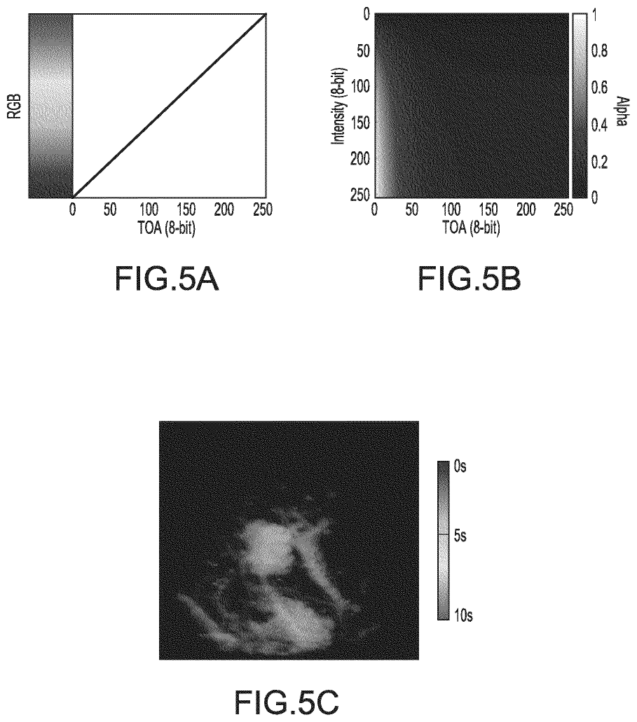

Turning to FIGS. 5A-5C assignment of color and opacity as a function of TOA to pixels on a display are described. As will become clearer as the present description continues, the 3D/4D volume image of FIG. 5C results from a combination of the more advantageous aspects of the methods described in FIGS. 3A-4C above. Various aspects and details of the method are common to those described in connection with representative embodiments of FIGS. 1-4C. These common aspects and details may not be repeated to avoid obscuring the presently described representative embodiment.

FIG. 5A shows a graph of color versus time of arrival (TOA) of a contrast agent according to a representative embodiment. As described above, according to one representative embodiment of the method (or algorithm), the assignment of a color to a particular data point varies linearly with the TOA, with the first arriving contrast data being coded red (R), whereas a particular data point from the source of ultrasound image data that arrives at a latest time is color coded blue (B). Again, the other colors of the visible spectrum between red and blue are assigned to data points arriving at times between the first (R) data point and the last (B) data point being assigned other colors (orange, yellow, green) along the spectrum of colors. Moreover, as noted above, the shading of the colors of the rainbow along the spectrum can be used to further discern the TOAs of the data points from the current CEUS scan by gradation of these shades with more shades providing greater distinction between TOA of data points represented by the color of the pixel. As such, the gradation of the shades of the colors of the visible spectrum between red and blue can be selected to identify the TOA of contrast agent with significant granularity and thus precision.

FIG. 5B shows the relationship between the assigned opacity value for each pixel in the resultant image of FIG. 5C with each pixel's having a color and an opacity/transparency value to characterize a time-based parameter (in this example TOA) of the contrast media dynamics. In the presently described representative embodiment, the opacity/transparency value at each pixel is based on both the TOA and the contrast intensity. As such, two parameters are used to determine the opacity at each pixel. Notably, the assigned color changes from comparatively opaque for the earliest arriving contrast agent and depicts the lowest transparency (T=1−α=0)/highest opacity (α=1), whereas later arriving contrast agent is assigned, for example a red, orange or yellow shade of color with decreasing opacity/increasing transparency as shown.

Moreover, the assignment of a contrast intensity value to an opacity/transparency value is done according to the relationship of FIG. 5B. Accordingly, in the presently described representative embodiment, the assigned color changes from comparatively opaque for the highest intensity contrast agent and depicts the lowest transparency (T=1−α=0)/highest opacity (α=1), whereas a lower intensity contrast agent is assigned, for example a red, orange or yellow shade of color with decreasing opacity/increasing transparency as shown. Generally, and as shown by example in FIG. 5B the relationship between the opacity, TOA and the intensity can be determined algorithmically, or by testing a large number of candidate shapes of the relationship to generate the results. The selected shapes are determined to provide sufficient image quality for improved diagnostic accuracy.

FIG. 5C is a depiction of a display of a CEUS imaging system of a representative embodiment depicting the color and opacity for each pixel of the display. Notably, the scale on the right of the drawing shows in gray scale the arrival time of data points from the CEUS with opacity values for each pixel being assigned based on the relationship of FIG. 5B. In this embodiment, by application of the relationships of FIGS. 5A and 5B to assign color and opacity of each data point, when depicted on a color display, the 3D TOA volume image has comparatively transparent colors on a surface of the anatomical part being viewed, making the discernment of internal structures of the anatomical part (e.g., lesion) better than in the image of FIGS. 3C and 4C.

The depiction of the display of the CEUS imaging system combines the benefit and advantages of the representative embodiments described in connection with FIGS. 3C and 4C. Specifically, the displayed image of FIG. 5C allows the visualization of the internal structure of a lesion, and the removal of surface obstacles from the image. While better illustrated in color, the image of FIG. 5C eliminates comparatively opaque colors (e.g., red—not shown) on a surface of the anatomical part being viewed, making the discernment of internal structures of the anatomical part (e.g., lesion) more difficult. Moreover, the image of FIG. 5C beneficially reducing residual clutter, early arriving contrast agent values, or background noise that all have comparatively low TOA values, and can create obstacles on the outer surfaces of the anatomical part being imaged, rendering review of the internal structure of the anatomical portion more difficult.

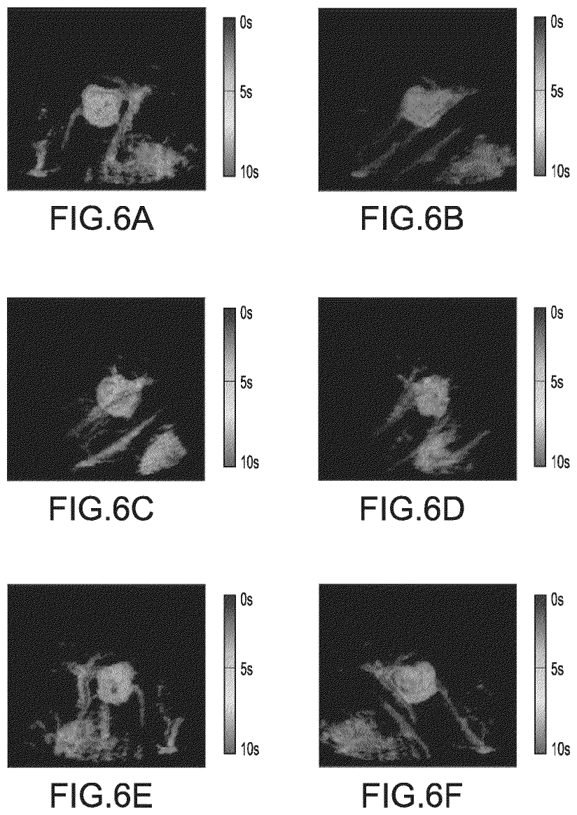

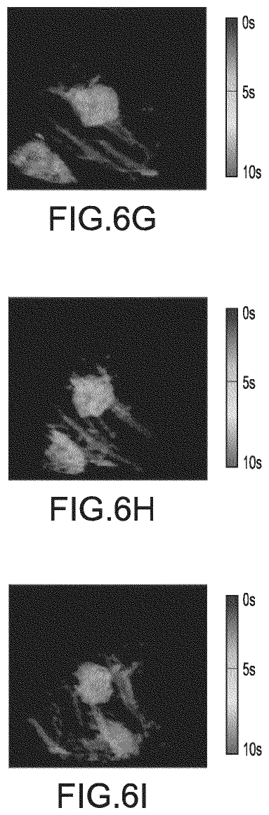

FIGS. 6A-6I are depictions of a display of a CEUS imaging system of a representative embodiment depicting the color and opacity of FIGS. 5A-5B for each pixel of the display according to a representative embodiment. Notably, the various views of FIGS. 6A-6I respectively show the anatomical part (e.g., lesion) from a variety of viewing angles as the image is rotated over 360° in approximately 40° increments. As such, each image of FIGS. 6A-6I is viewed at an angle that is rotated clockwise by an angle 40° from the previous view. As such, in regions where it is useful to see “through” the image, the opacity values assigned to the pixels that could block the image are made transparent as described below. By contrast, the opacity values assigned to the pixels on a surface of the anatomical part are assigned a comparatively high opacity value so the portion of the anatomical image to be shown can be viewed from a particular angle/vantage point. The displayed images of FIG. 6A-6I allow the visualization of the internal structure of a lesion, and the removal of surface obstacles from the image. While better illustrated in color, images of FIG. 6A-6I eliminates comparatively opaque colors (e.g., red—not shown) on a surface of the anatomical part being viewed, making the discernment of internal structures of the anatomical part (e.g., lesion) more difficult. Moreover, the images of FIG. 6A-6I of FIG. 5C beneficially reduce residual clutter, early arriving contrast agent values, or background noise that all have comparatively low TOA values, and can create obstacles on the outer surfaces of the anatomical part being imaged, rendering review of the internal structure of the anatomical portion more difficult.

As will be appreciated by one of ordinary skill in the art having the benefit of the present disclosure, devices, systems and methods of the present teachings provide the transmission of echo image data from an ultrasound device. For example, compared to known methods and systems, various aspects of a protocol including the beginning, duration and termination of a step in the protocol can be facilitated during the generation of the protocol, or during implementation of the protocol, or both. Moreover, errors that can result from human interaction with an imaging system can be reduced thereby reducing the need to repeat procedures, and reducing the time required to complete an imaging procedure. Notably, these benefits are illustrative, and other advancements in the field of medical imaging will become apparent to one of ordinary skill in the art having the benefit of the present disclosure.

Although methods, systems and components for implementing imaging protocols have been described with reference to several exemplary embodiments, it is understood that the words that have been used are words of description and illustration, rather than words of limitation. Changes may be made within the purview of the appended claims, as presently stated and as amended, without departing from the scope and spirit of the protocol implementation of the present teachings. The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A system for providing ultrasound images, comprising:
a source of ultrasound signal data from a contrast-enhanced ultrasound (CEUS) examination;
a processor; and
a tangible, non-transitory computer-readable medium that stores instructions, which when executed by the processor cause the processor to: determine a color value of a contrast agent as a function of a selected time-based parameter for each pixel; determine an opacity value of the contrast agent for each pixel; and a display in communication with the processor and configured to display the color value and the opacity value for each pixel.

2. The system of claim 1, wherein the determined opacity value is a function of the selected time-based parameter for each pixel.

3. The system of claim 1, wherein the determined opacity value is a function of an intensity of the contrast agent for each pixel.

4. The system of claim 1, wherein the determined opacity value is a function of the selected time-based parameter and an intensity of the contrast agent for each pixel.

5. The system of claim 1, wherein the selected time-based parameter is one of a time of arrival (TOA) of the contrast agent, a time to peak of the contrast agent, a wash-in rate of the contrast agent, a rise time of the contrast agent, a peak intensity of the contrast agent, a wash-out rate of the contrast agent, a mean transit time of the contrast agent, a fall time of the contrast agent, and an area under a curve of the contrast agent for each pixel.

6. The system of claim 1, wherein the opacity values are a function of the CEUS intensity, or temporal statistics of the CEUS intensity.

7. The system of claim 1, wherein the instructions further cause the processor initially to pre-process an input of contrast-enhanced ultrasound (CEUS) data for each pixel.

8. The system of claim 7, wherein the pre-process comprises one or more of: normalizing CEUS data, reducing background noise to increase a contrast-to-noise ratio (CNR); and rejecting clutter.

9. The system of claim 8, wherein the CEUS data comprises the contrast intensity for each pixel.

10. A tangible, non-transitory computer-readable medium that stores instructions, which when executed by a processor cause the processor to: determine a color value of a contrast agent as a function of a selected time-based parameter for each pixel; determine an opacity value of the contrast agent for each pixel; and display the color and opacity value for each pixel.

11. The tangible, non-transitory computer-readable medium of claim 10, wherein the determined opacity value is a function of the selected time-based parameter for each pixel.

12. The tangible, non-transitory computer-readable medium of claim 10, wherein the opacity value is determined opacity value is a function of an intensity of the contrast agent for each pixel.

13. The tangible, non-transitory computer-readable medium of claim 10, wherein the determined opacity value is a function of the selected time-based parameter and an intensity of the contrast agent for each pixel.

14. The tangible, non-transitory computer-readable medium of claim 10, wherein the selected time-based parameter is one of one of a TOA of the contrast agent, a time to peak of the contrast agent, a wash-in rate of the contrast agent, a rise time of the contrast agent, a peak intensity of the contrast agent, a wash-out rate of the contrast agent, a mean transit time of the contrast agent, a fall time of the contrast agent, and an area under a curve of the contrast agent for each pixel.

15. The tangible, non-transitory computer-readable medium of claim 10, wherein the instructions further cause the processor initially to pre-process an input of contrast-enhanced ultrasound (CEUS) data for each pixel.

16. The tangible, non-transitory computer-readable medium of claim 15, wherein the pre-process comprises one or more of: normalizing CEUS data, reducing background noise to increase a contrast-to-noise ratio (CNR); and rejecting clutter.

17. The tangible, non-transitory computer-readable medium of claim 16, wherein the CEUS data comprises the contrast intensity for each pixel.

18. A method of providing ultrasound images, the method comprising:
determining a color value of a contrast agent as a function of a selected time-based parameter for each pixel;
determining an opacity value of the contrast agent for each pixel; and
displaying the color value, and the opacity value for each pixel.

19. The method of claim 18, wherein the determining the opacity value further comprises determining the opacity value as a function of the selected time-based parameter for each pixel.

20. The method of claim 18, determining the opacity value further comprises determining the opacity value as a function of an intensity of the contrast agent for each pixel.

21. The method of claim 18, wherein the determining the opacity value further comprises determining the opacity value as a function of the selected time-based parameter and an intensity of the contrast agent for each pixel.

22. The method of claim 18, wherein the selected time-based parameter is one of a TOA of the contrast agent, a time to peak of the contrast agent, a wash-in rate of the contrast agent, a peak intensity of the contrast agent, and an area under a curve of the contrast agent for each pixel.
